(19) United States
(12) Patent Application Publication
(10) Pub. No.: US 2004/0220282 A1
(43) Pub. Date: Nov. 4, 2004

(54) Animal repellent formulation and method for preparing the same

(76) Inventor: Gregory A. La Torre, Colonia, NJ (US)

Correspondence Address: Gregory A. La Torre, P.O. Box 7241, Colonia, NJ, 07067, US

(21) Appl. No.: 10/731,658

(22) Filed: Dec. 9, 2003

Related U.S. Application Data
(60) Provisional application No. 60/446,390, filed on Feb. 12, 2003.

(51) Int. Cl. A01M 5/00; A61K 31/12

(52) U.S. Cl. 514/690

(57) ABSTRACT

An animal repellent composition used for protecting vegetation from the destructive browsing habits of animals. Particularly deer, rabbits & Canadian geese that are repelled both by the smell and taste of this formulation. The composition is comprised of Mentha Piperita (Peppermint oil), hydrogenated vegetable oil, garlic oil, white pepper, and gum arabic as a thickener/emulsifier and natural sticking/adherent agent. Additionally, whole or powdered eggs, vinegar and water can be added to this formulation. This composition would then be applied in the form of spraying on all vegetation needing protection from above said animals.

### BACKGROUND OF THE INVENTION

While visiting friends in central Pennsylvania, I was suffering with severe nasal congestion. As a home remedy I was inhaling Mentha Piperita (Peppermint oil) soaked in a towel to relieve the symptoms of the congestion. During this time and while observing the intense deer & rabbit overpopulation problem and the damage created by these animals, it occurred to me with my background as an animal enthusiast and knowing how sensitive these animals sense of smell and taste are, that the strong burning sensation that occurs while smelling or inhaling Peppermint oil would repel these animals particularly, deer, rabbits, and Canadian geese when applied to the vegetation with a folier spray.

I prepared a mixture of 1 ounce of Mentha Piperita (Peppermint oil), 1 ounce of vegetable oil, 1 egg and water, and applied it to the vegetation using a commercial spray device. The repelling of the deer & rabbits was immediate and the initial application remained effective for approximately 60 days.

### SUMMARY OF THE INVENTION

The object of this invention is to provide an all natural biodegradable animal repellent (particularly deer, rabbits, and Canadian geese) composition which contains no synthetic ingredients that can be safely applied to all plantings in need of protection from the destructive browsing habits of deer, rabbits and Canadian Geese.

Furthermore, the above animal repellent composition is pleasant smelling.

Furthermore, the above animal repellent composition won't wash off during periods of rain or watering.

Furthermore, the above said animal repellent composition is safer for humans and animals compared to products containing potentially harmful synthetic ingredients.

Furthermore, to offer an organic animal repellent composition that stimulates re growth to plantings previously damaged by above said animals.

Furthermore, that the above said animal repellent composition will not burn, stain, or leave an unsightly residue when applied in the form of a folier spray.

### DETAILED DESCRIPTION OF THE PREFERRED EMBODIMENT

The Mentha Piperita (Peppermint oil) based animal repellent composition include the following ingredients: 20 ounces (11.25%) of Mentha Piperita (Peppermint Oil) 10 ounces (7.5%) of hydrogenated vegetable oil, 5 ounces (3.75%) of garlic oil, 10 teaspoon (0.0035 to 0.0075 percent) of white pepper, 10 teaspoons of Gum Arabic, 30 (47%) large eggs, or 25 oz of powdered eggs (10) Additionally 25 ounces (25%) of white vinegar and enough water (approximately 40 to 48 oz) to produce 1 gallon of concentrate.

The animal repellent concentrate would then be mixed with water at a ratio of 12.8 oz of concentrate to 115 oz of water to make 1 gallon of a ready to use mixture.

The preferred method in which the animal repellent ready to use mixture would be utilized would be to thoroughly apply a mist or spray from a commercial or folier sprayer to vegetation protecting them from the destructive eating habits of, deer, rabbits, and Canadian geese. Generally one application of the animal repellent composition remains effective for a period of three to four months.

One gallon of the ready to use mixture would cover approximately 4000 sq. Ft.

The strong taste and smell of Mentha Piperita (Peppermint oil) and the burning sensation that occurs while either breathed in through the nose or when attempted To be eaten by deer, rabbits, or Canadian Geese are found to be extremely offensive to these animals. Furthermore, any plantings and vegetation sprayed with the animal repellent invention were no longer eaten by these animals for periods of up to three or four months.

### EXAMPLE 1

Two piles of apples were placed approximately 25 feet apart, one pile was sprayed with the animal repellent invention while the other was not. Within 1 hour the test area was visited by 17 deer, The untreated apples were quickly consumed, while the apples treated with the animal repellent compositon remained uneaten.

Deer continued visiting this test area twice a day for approximately 30 days and never ate the apples treated with the animal repellent invention.

### EXAMPLE 2

A group of deer were visiting and eating the buds from a group of untreated Azaleas on a daily basis. The Azaleas were then sprayed with the animal repellent invention and the results were immediate, the deer continued to pass through that immediate area on a daily basis without eating any of the remaining treated Azalea buds. About 45 days later and after a recent snow storm there was evidence that deer removed the snow from the top of the treated Azaleas in an attempt to eat the remaining buds, but the buds remained uneaten.

The two test areas used in examples 1 and 2 were intensely surveyed over quite a few months with various tests performed under a variety of conditions. It was observed, further damage done by browsing deer and rabbits was nonexistent after these areas were treated with the above said animal repellent invention.

It is to be understood, that the present animal repellent invention described including the Preferred embodiment and all examples given are words of description rather than limitations, and that changes may be made without thereby departing from the scope of the present invention as defined in the ensuing claims and the equivalents thereof.

What is claimed is:

1. An all natural Mentha Piperita (Peppermint oil) based animal repellent concentrate used to protect vegetation, plantings, fruits and vegetables, and trees from the destructive eating habits of deer, rabbits & Canadian geese. With a composition comprised of 10 to 15 ounces of Mentha Piperita (Peppermint oil), 5 to 10 ounces of hydrogenated vegetable oil, 1 to 5 ounces of Garlic oil and 10 to 20 grams of powdered white pepper and sufficient water to total 128 ounces of concentrate.

2. I claim the use of the ingredients in claim #1 with the addition of 20 to 30 ounces of white vinegar.

3. I claim the use of the ingredients in claim #2 with the addition of 20 to 30 whole eggs or 15 to 25 ounces of powdered whole eggs.

4. I claim the use of the ingredients in claim #3 with the addition of gum arabic as a thickener and a natural sticking/adherent agent.

5. I claim the use of the ingredients in #3 with the addition of guar gum as a thickener and a natural sticking/adherent agent.

6. I claim the animal repellent invention as defined in claim #1 wherein the animal is deer.

7. I claim the animal repellent invention as defined in claim #1 wherein the animal is rabbits.

8. I claim the animal repellent invention as defined in claim #1 wherein the animal is Canadian geese.
